(19) United States
(12) Patent Application Publication
(10) Pub. No.: US 2010/0262034 A1
(43) Pub. Date: Oct. 14, 2010

(54) GAS COMPONENT DETECTION DEVICE

(76) Inventors: Tomohisa KAWATA, Osaka-shi (JP); Mikihiro Yamanaka, Osaka-shi (JP); Keita Hara, Osaka-shi (JP)

Correspondence Address: NIXON & VANDERHYE, PC, 901 NORTH GLEBE ROAD, 11TH FLOOR, ARLINGTON, VA, 22203, US

(21) Appl. No.: 12/759,116

(22) Filed: Apr. 13, 2010

(30) Foreign Application Priority Data
Apr. 13, 2009 (JP) 2009-096694(P)

(51) Int. Cl. A61B 5/097; G01N 30/02

(52) U.S. Cl. 600/532; 73/23.39

(57) ABSTRACT

There is provided a gas component detection device including: a gas introduction unit for introducing a specimen gas; a gas separation unit connected to the gas introduction unit; a flow path switching unit connected to the gas separation unit and having a plurality of connection flow paths for switching a flow path that is connected to the gas separation unit to any one of the plurality of connection flow paths; and a gas detection unit provided in at least one of the plurality of connection flow paths. The gas separation unit is preferably formed of a chromatography column having therein a flow path having a width and depth of micro order.

This nonprovisional application is based on Japanese Patent Application No. 2009-096694 filed on Apr. 13, 2009 with the Japan Patent Office, the entire components of which are hereby incorporated by reference.

### BACKGROUND OF THE INVENTION

1. Field of the Invention

The present invention relates to gas component detection devices capable of measuring a gas component concentration in a specimen gas with high precision.

2. Description of the Background Art

Japan's population is shrinking and graying with decrease of birthrate, and it is estimated that in the years of 2013 and 2035, Japan's aging population rates will be 25.2% and 33.7%, respectively, i.e., a super-aging society is foreseen to come, in which one out of four and three Japanese people, respectively, will be the aged over 65 years old. In anticipation of the coming aging society, metabolic syndrome is increasingly discussed in recent years as a social problem. Accordingly, preventive medicine is gaining attention. The promotion of the preventive medicine decreases patients and hence medical expenses can be reduced in the present day suffering increased medical expenses and the collapsing medical insurance system.

The promotion of the preventive medicine requires a system capable of health care making use of health information measured with a household instrument. Indexes allowing individuals to easily obtain information of their health conditions include blood pressure, blood, urine, sweat, saliva, exhaled breath and other biological samples. Such biological samples contain multiple substances varied in numerical value depending upon diseases or their symptoms, such as blood sugar level when the biological sample is blood. Measuring how such substances vary in amount provides a large possibility that individuals can obtain information of their own health conditions by themselves, and constantly measuring how the substances vary in amount allows health care, and early detection of diseases.

Among the above biological samples, exhaled breath is a biological sample suitable for constant measurement, as it includes a plurality of types of substances varied in numerical value depending upon diseases or their symptoms, can be sampled and measured quickly and conveniently, and includes substances to be measured in the form of gas and hence can be measured non-invasively with less physical damage. Furthermore, it is known that patients with lung cancer exhale breath having components different from those of the able-bodied, and accordingly, exhaled breath components can be measured to detect cancer to some extent. Exhaled breath thus includes a large amount of information on diseases and is accordingly, actively studied in recent years.

For example, exhaled breath contains nitrogen monoxide and carbon monoxide, which have a high correlation with lung diseases and are detected in high concentrations in the exhaled breathes of patients with asthma, chronic obstructive pulmonary disease (COPD) and the like. Patients with dyspepsia, duodenal ulcer or other gastrointestinal diseases provide exhaled breath containing hydrogen. Ethane, pentane and the like have a high correlation with oxidative stress, and are detected from patients with lipid oxidation, asthma, bronchitis, and the like. Furthermore, acetone in exhaled breath has conventionally been positioned as an index of defective saccharometabolism and is known to be contained in the exhaled breathes of the diabetics in large amounts. Acetone in exhaled breath is produced from fat (fatty acid) and protein (amino acid), and generated when a living body is in inanition (as it is in extreme hunger, on a fast or the like and thus incapable of athrocytosis) or has serious diabetes. Furthermore, while acetone is an end product of fat metabolism as described above, it is also an index of metabolic activity in a body, and it has been reported that there is a correlation between acetone in exhaled breath and body fat reduction. When blood glucose is consumed on a diet, by sports or the like and thus running out, stored body fat is used as energy and accordingly, fat is metabolized. Fat metabolism process generates ketone substances such as acetoacetic acid, hydroxybutyric acid and acetone in blood. Acetoacetic acid and hydroxybutyric acid are reused in an organ other than liver and acetone is discharged with exhaled breath via lung. Acetone is thus produced in a body fat burning process and discharged into exhaled breath, and the measurement of acetone in exhaled breath directly provides information on the extent of body fat burning. Thus, exhaled breath measurement can make it possible to obtain disease information and to provide health guidance.

A plurality of components in exhaled breath are measured in concentration in a method conventionally known as follows: gas chromatography is employed to separate each component which is then detected with a detector such as a thermal conductivity detector, a flame ionization detector, an electron capture detector or a mass spectrometer on the ppb-ppt level with high sensitivity. However, conventional measuring instruments are large in size and weight and also expensive, and require the user's familiarization of their operation methods. They are thus not practical for widespread use in every household and would not be effective for promoting preventive medicine.

The above issue can be addressed by a measuring instrument disclosed for example in Japanese Patent Laying-Open No. 2003-057223 as a palm-top, microminiature, ultralight gas analyzer.

### SUMMARY OF THE INVENTION

As disclosed in Japanese Patent Laying-Open No. 2003-057223, the gas analyzer employs a gas detection means implemented as a non-selective semiconductor gas sensor responding to a variety of types of gases. Generally, such a semiconductor gas sensor has a small return rate. When the gas analyzer is used to measure a specimen gas containing a plurality of components, the semiconductor gas sensor is exposed to each component separated by a microcolumn, and whenever the sensor is exposed to each component, the sensor will respond. In doing so, however, the sensor returns incompletely, and may not be able to detect each component's concentration accurately.

Accordingly, the present invention contemplates a gas component detection device having a miniature and simple structure and capable of measuring the concentration of a gas component to be measured with high precision even if a specimen gas has a plurality of gas components.

The present gas component detection device includes: a gas introduction unit for introducing a specimen gas; a gas separation unit connected to the gas introduction unit; a flow path switching unit connected to the gas separation unit and having a plurality of connection flow paths for switching a flow path that is connected to the gas separation unit to any one of the plurality of connection flow paths; and a gas detection unit provided in at least one of the plurality of connection flow paths.

The present gas component detection device may further include a control unit for controlling an operation performed by the flow path switching unit for switching. The control unit preferably includes a storage unit having any preset time stored therein to control in accordance with the preset time the operation performed by the flow path switching unit for switching. Alternatively, the control unit may include a storage unit having stored therein any one or two or more preset gas components and a period(s) of time elapsing after any one or two or more gas components are introduced through the gas introduction unit before any one or two or more gas components are detected by the gas detection unit. In that case, the operation performed by the flow path switching unit for switching is controlled in accordance with the period(s) of time of any one or two or more gas components selected.

The gas separation unit is preferably formed of a chromatography column having therein a flow path having a width and depth of micro order. The chromatography column can have the flow path in one of a meandering form, a squarely angled spiral form and a circular spiral form. Furthermore, the gas detection unit is preferably a movable gas sensor.

The present gas component detection device can measure the specimen gas that is exhaled breath. In that case, the gas introduction unit preferably has a port receiving and introducing the exhaled breath. The specimen gas includes acetone for example.

The present invention can thus provide a gas component detection device having a miniature and simple structure and capable of measuring the concentration of a gas component to be measured with high precision even if the specimen gas has a plurality of gas components. The present gas component detection device as above is useful as a component detection device capable of conveniently measuring the concentration of a particular component in exhaled breath. The present invention can thus provide a gas component detection device that is effective in promoting preventive medicine, small in size, and suitable for personal use.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 schematically shows the present gas component detection device in a preferable example.

FIGS. 2A-2C are schematic plan views of examples in geometry of an internal flow path formed in a microcolumn.

FIGS. 3A-3D schematically show examples of a switching means that can be used suitably for a flow path switching unit.

FIGS. 4A and 4B are schematic plan views of examples of how in the present gas component detection device the flow path switching unit is arranged.

FIGS. 5A-5D are schematic cross sections showing examples of where in a flow path a gas sensor is positioned.

FIG. 6 schematically shows the present gas component detection device in another preferable example.

FIG. 7 schematically shows the present gas component detection device in still another preferable example.

FIGS. 8A and 8B show chromatograms indicating a result of detecting a gas component with a gas component detection device of Example 1.

### DESCRIPTION OF THE PREFERRED EMBODIMENTS

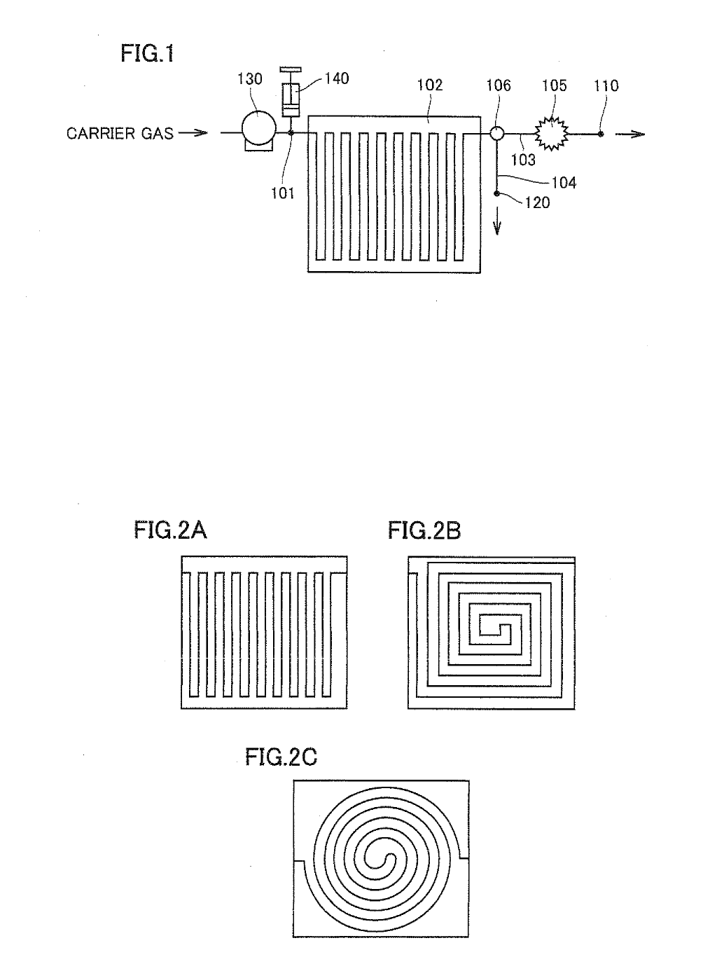

FIG. 1 schematically shows the present gas component detection device in a preferable example. The gas component detection device as shown in FIG. 1 basically includes a gas introduction unit 101 for introducing a specimen gas, a gas separation unit 102 connected to gas introduction unit 101, a flow path switching unit 106 connected to gas separation unit 102 and having a plurality of connection flow paths (a first flow path 103 and a second flow path 104) for switching a flow path that is connected to gas separation unit 102 to any of the plurality of connection flow paths, and a gas detection unit 105 provided in first flow path 103. More specifically, the gas component detection device basically includes: gas introduction unit 101 for introducing a specimen gas into the device; gas separation unit 102 connected to gas introduction unit 101; first flow path 103 connected to gas separation unit 102 for guiding the specimen gas that has passed through gas separation unit 102 to a first gas discharging unit 110; second flow path 104 branched from first flow path 103 for guiding the specimen gas that has passed through gas separation unit 102 to a second gas discharging unit 120; gas detection unit 105 provided in first flow path 103; and flow path switching unit 106 switching a flow path that is connected to gas separation unit 102 to any one of first flow path 103 and second flow path 104. Flow path switching unit 106 is provided between gas separation unit 102 and gas detection unit 105. From flow path switching unit 106 to second gas discharging unit 120 a flow path extends, which is second flow path 104. Furthermore, a portion of first flow path 103 configures a flow path extending from flow path switching unit 106 to first gas discharging unit 110. Gas detection unit 105 is provided in first flow path 103 between flow path switching unit 106 and first gas discharging unit 110.

Furthermore, the gas component detection device as shown in FIG. 1 further includes a pump 130 for introducing a carrier gas into gas separation unit 102.

The gas component detection device as shown in FIG. 1 including flow path switching unit 106 between gas separation unit 102 and gas detection unit 105 allows a specimen gas that has passed through gas separation unit 102 to be introduced into first flow path 103 or second flow path 104, as switched as desired. While a specimen gas having passed through gas separation unit 102 is separated into a plurality of components, only a particular component thereof (i.e., a target component to be measured in concentration) can be guided to and detected by gas detection unit 105 provided in first flow path 103. The present gas component detection device can thus guide only a target component to the gas detection unit and detect the target component, and the gas detection unit is not exposed to a component other than the target component. When a detector (a gas sensor or the like) that has a relatively small return rate and responds to a plurality of gas components is used as the gas detection unit, it is not affected by components other than the target component, and the present gas component detection device can thus measure the target component's concentration satisfactorily reproducibly with high precision.

The gas component detection device as shown in FIG. 1 is operated to measure the concentration of the target component in the specimen gas, by way of example as follows: Initially, pump 130 is actuated and a carrier gas serving as a mobile phase (for example, an inert gas such as helium, or air or the like) is communicated to gas separation unit 102. Then, in that condition, for example, a syringe 140 having the specimen gas accommodated therein is inserted into gas introduction unit 101 to introduce the specimen gas into the device. The specimen gas thus passes together with the carrier gas through a flow path that connects gas introduction unit 101 and gas separation unit 102 together, and the specimen gas thus enters gas separation unit 102 and has its components separated therein. When gas separation unit 102 is a chromatography column, the specimen gas introduced into gas separation unit 102 is repeatedly adsorbed and desorbed between a stationary phase (adsorbent) provided in the column and the mobile phase, and as its adsorbability/desorbability depends on its components' types, it has its components separated accordingly. More specifically, a component having high adsorbability to the stationary phase moves in the gas separation unit slowly and a component having low adsorbability to the stationary phase moves in the gas separation unit fast, and accordingly, a component poorer in adsorbability to the stationary phase is discharged externally from the gas separation unit faster. A period of time that passes after the specimen gas is introduced into the device before it is detected by the gas detection unit is referred to as a retention time. A retention time indicates a value unique to a substance. Thus a retention time can be referenced to identify a component.

Each component in the specimen gas separated as the specimen gas has passed through gas separation unit 102 is guided externally from gas separation unit 102 successively in accordance with its retention time. In doing so, before a target component's retention time arrives, flow path switching unit 106 is adjusted to connect gas separation unit 102 to second flow path 104. Thus a component having a shorter retention time than the target component is passed through second flow path 104 and thus discharged from second gas discharging unit 120. Then when the target component's retention time comes close, flow path switching unit 106 is switched to connect gas separation unit 102 to first flow path 103. The target component thus passes through first flow path 103 and arrives at gas detection unit 105 and is detected thereby. The gas which has passed through gas detection unit 105 is discharged from first gas discharging unit 110. Once the target component has been detected, flow path switching unit 106 is again switched to connect gas separation unit 102 to second flow path 104 and disconnect gas detection unit 105. Thus only the target component can be guided to gas detection unit 105 and detected thereby. Hereinafter, each component of the present gas component detection device will be described more specifically.

<Gas Introduction Unit>

The gas introduction unit is a component for introducing a specimen gas into the device and it is not limited to any particular structure as long as it can introduce the specimen gas into the device. The gas introduction unit in the simplest form may be an end per se of a flow path extending from the gas separation unit. Furthermore the gas introduction unit may have a configuration that can connect a specimen gas accommodating means, such as a syringe accommodating the specimen gas, to a flow path extending to the gas separation unit, as indicated in FIG. 1 by way of example. Such configuration can for example be the configuration in which a glass capillary tube is connected to an introduction port of the gas separation unit and an opening for receiving the syringe is connected to the capillary tube using a reducing union.

Furthermore, when the present gas component detection device is used as an exhaled breath component detection device for measuring a particular component of exhaled breath in concentration, it is preferable that the gas introduction unit have a port receiving and introducing exhaled breath, such as a mouth piece, connected to a flow path that extends to the gas separation unit and allowing a user to put his/her mouth at the port and blow exhaled breath directly thereinto.

The flow path extending from the gas introduction unit to the gas separation unit preferably includes a gas flow rate control means to introduce a specimen gas at a predetermined flow rate to the gas separation unit. The gas flow rate control means is for example a gas sampler.

<Gas Separation Unit>

The gas separation unit is a component for separating a variety of types of components in a specimen gas introduced from the gas introduction unit. A chromatography column can suitably be used as the gas separation unit, and the chromatography column is not limited to any particular column, and may be a capillary column or a packed column filled with an adsorbent. Inter alfa, a microcolumn is suitably used, as it can help to reduce the device in size and weight. When the capillary column or the packed column filled with the adsorbent is used, it is necessary to use a large incubator to control the column in temperature, and it is thus difficult to reduce the device in size. The microcolumn for example means a chromatography column in the form of a chip including a substrate such as a Si wafer and a fine flow path provided in the substrate and having a width and depth of micro order. In the present invention the microcolumn is not limited to any particular size and for example can have longitudinal and lateral dimensions of several millimeters to several tens centimeters and a thickness of several millimeters to several centimeters approximately. The microcolumn may include a temperature control means.

The microcolumn can be prepared for example as follows: Initially, a continuous groove is formed on a surface of a substrate such as a Si wafer by etching with a photolithography technique. Then the etched substrate and a glass plate are hermetically bonded together by anodic bonding or a similar method such that the substrate has the grooved surface facing the glass plate. Subsequently, an unmodified glass capillary is attached to one end of an internal flow path formed and a solution of stationary phase is introduced into the internal flow path of the microcolumn, and then a solvent is removed to modify an internal wall of the internal flow path of the microcolumn with the stationary phase.

The internal flow path in the microcolumn can have a width and a depth (or height) each of approximately 100-300 μm for example. In determining the width and depth of the internal flow path in the microcolumn, it is preferable that the type of the target component, the flow rate of the specimen gas introduced into the microcolumn, and the like are taken into consideration. A liquid phase constituting the stationary phase secured to the internal wall of the internal flow path in the microcolumn is not particularly limited, and can for example be paraffinic hydrocarbon, fluorine containing-oil, monoesters, polyesters, alcohols, ethers, polyglycols, amides, amine acid, nitriles, nitro compounds, methylsilicone, methylphenylsilicone, methylphenylvinylsilicone, trifluoropropylsilicone, cyanoalkylmethylsilicone, cyanopropylphenylsilicone, sulfur compounds, phosphate ester, salts, chlorinated organic acid compounds or the like. Furthermore, a filler of a carrier coated with liquid phase can also be introduced into the flow path. The carrier can be diatomaceous earth, fluororesin, crystal, glass bead, terephthalic acid, porous polymer, carbon, alumina, activated charcoal or the like. In determining the type of the stationary phase, it is preferable that the type of the specimen gas, the type of the target component and the like are taken into consideration.

FIGS. 2A-2C are schematic plan views of examples in geometry of an internal flow path formed in a microcolumn. As shown in FIGS. 2A-2C, the internal flow path may have a meandering form (FIG. 2A), a squarely angled spiral form (FIG. 2B) or a circular spiral form (FIG. 2C), or the like. For example, for an area of 2 cm square having a flow path of a width of 100 μm at intervals of 100 μm, the circular spiral form as shown in FIG. 2C allows a flow path to have a total length of approximately 1.5 m. In contrast, the meandering flow path (FIG. 2A) and the squarely angled spiral flow path (FIG. 2B) allow a total length of approximately 2 m in distance and have a difference in distance of approximately 0.5 m from the circular spiral flow path. This difference has larger values for larger areas (of a surface of the substrate).

In general, the circular spiral flow path is more advantageous than the squarely angled spiral and meandering flow paths as the former provides a smaller gas pressure loss. Accordingly, if the specimen gas contains a target component having a retention time which is significantly different from that of another component to be separated, the use of the circular spiral microcolumn is advantageous in more efficient separation of the components since the flow rate of a carrier gas containing the specimen gas can be increased. The squarely angled spiral microcolumn or the meandering microcolumn has a tendency to have a more excellent separation performance than the circular spiral microcolumn, as the former can provide a flow path having a larger total length and a relatively larger pressure loss than the latter. As such, when a specimen gas has a component less separatable from a target component, the squarely angled spiral microcolumn or the meandering microcolumn is preferably used.

The internal flow path in the microcolumn has one opening (a column inlet port) and the other opening (a column outlet port), which are not limited to any particular positional relationship. It is preferable, however, that one and the other openings be provided at opposite sides, respectively, as shown in FIGS. 2A-2C. This configuration is advantageous in terms of modifying the stationary phase of the column, arranging the column in the device, and the like. For the circular and squarely angled spiral flow paths, the openings having a positional relationship as described above can be achieved by forming the flow path from a peripheral edge of the substrate toward the center of the substrate spirally and subsequently folding back the flow path at the center of the substrate to extend from the center of the substrate toward a peripheral edge of the substrate spirally, as shown in FIGS. 2B and 2C.

<Flow Path Switching Unit, and First and Second Flow Paths>

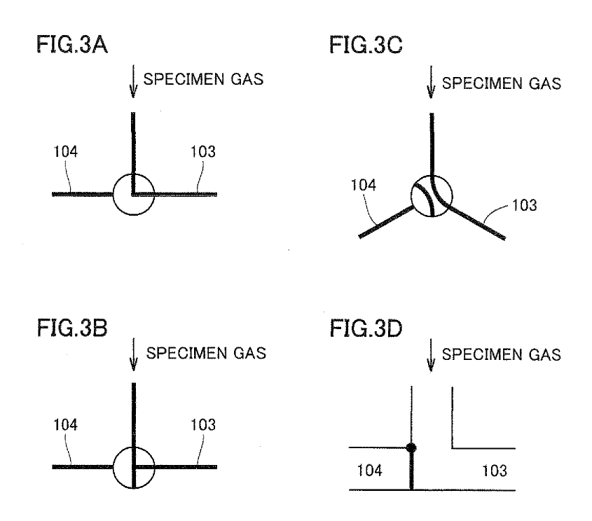

The flow path switching unit is a component for switching a flow path that is connected to the gas separation unit (e.g., a column outlet port of a microcolumn) to a first flow path or a second flow path. FIGS. 3A-3D schematically show examples of a switching means that can be used suitably for the flow path switching unit. FIGS. 3A-3D all show gas separation unit 102 connected to first flow path 103. The flow path switching unit may be a switching means in the form of a valve, as shown in FIGS. 3A-3C, or a switching means in a valve system, as shown in FIG. 3D. Furthermore, the switching means in the form of the valve may be a two-way valve (FIG. 3A), a three-way valve (FIG. 3B), or a four-way valve (FIG. 3C). The flow path switching unit is provided between the gas separation unit and the gas detection unit, as described above.

The first flow path is a flow path for guiding a specimen gas that has passed through the gas separation unit to the first gas discharging unit. The second flow path is a flow path for guiding a specimen gas that has passed through the gas separation unit to the second gas discharging unit, and the second flow path extends from the flow path switching unit to the second gas discharging unit.

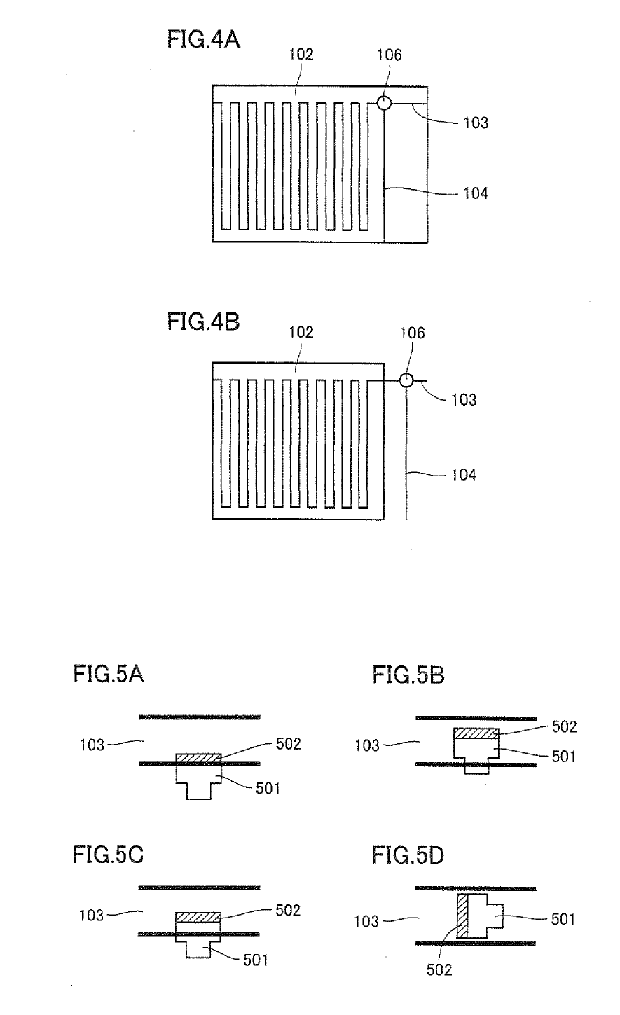

When the gas separation unit is a microcolumn, the flow path switching unit may be incorporated in a substrate configuring the microcolumn, for example as shown in FIG. 4A. The gas component detection device can further be reduced in size. In that case, the first and second flow paths connected to the flow path switching unit can be internal flow paths formed in the substrate configuring the microcolumn. Furthermore, the flow path switching unit may be provided in the device at a location different from the substrate configuring the microcolumn, and may be connected to the column outlet port of the microcolumn by a flow path, as shown in FIG. 4B.

The flow path switching unit may be operated to manually or automatically switch connecting to the first flow path or the second flow path. If the flow path switching unit is operated manually, it is recommendable that a target component's retention time (a period of time elapsing after the target component is introduced into the device before the target component is detected by the gas detection unit) be previously obtained and when the target component's retention time is approaching or immediately before the target component's retention time arrives the flow path switching unit be switched to disconnect the gas separation unit from the second flow path and connect the gas separation unit to the first flow path including the gas detection unit. Furthermore, preferably, after the target component is detected, the flow path switching unit is switched to again connect the gas separation unit to the second flow path to prevent exposing the gas detection unit to components other than the target component.

If the flow path switching unit is operated to automatically switch connecting to the first flow path or the second flow path, the gas component detection device further includes a control unit controlling the switching operation by the flow path switching unit. The control unit preferably includes a storage unit having any preset time stored therein to automatically control the switching operation by the flow path switching unit in accordance with the preset time. The control unit more preferably includes a storage unit having stored therein any one or two or more preset gas components that can be a target component(s) and its/their respective retention time(s). When any one of the gas component(s) stored in the storage unit (typically, a target component to be measured) is selected, the control unit automatically controls the switching operation by the flow path switching unit in accordance with the retention time of the selected gas component.

More specifically, for example, when the selected gas component's retention time is approaching or immediately before the selected gas component's retention time arrives, the gas separation unit is connected to the first flow path, as switched by the flow path switching unit automatically. Connecting to the first flow path to detect the target component and connecting to the second flow path after the target component has been detected may be switched as automatically controlled in accordance with the retention time of the target component stored in the storage unit. Furthermore, to accommodate changing a target component, it is preferable that the control unit include a storage unit having stored therein two or more gas components that can be target components and their respective retention times, and timing for switching by the flow path switching unit can be varied in accordance with a selected gas component's retention time.

The control unit including the storage unit can for example be a personal computer, or a mobile terminal such as a mobile phone, a PHS or a PDA.

<Gas Detection Unit>

The gas detection unit is a component for detecting a target component and in the present invention a gas sensor is used for it. In the present invention, the flow path switching unit allows a target component to be selectively guided to the gas detection unit, and accordingly, the gas detection unit can for example be a non-selective semiconductor sensor in which a sensing portion for sensing a chemical substance is configured of SnO₂, ZnO or the like. Alternatively, the gas detection unit can be a nanostructure sensor in which a sensing portion is configured of carbon nanotube having a surface modified with a metal complex to detect a target component with higher sensitivity. The semiconductor and nanostructure sensors can measure the concentration of a target component in a specimen gas based on variation in voltage of the constant resistance in the sensor as the sensing portion is exposed to the target component. The metal complex can for example be cobalt (II) phthalocyanine, iron (II) phthalocyanine, copper (II) phthalocyanine, manganese (II) phthalocyanine or the like.

FIGS. 5A-5D are schematic cross sections showing examples of where in a flow path a gas sensor is positioned. In first flow path 103 a gas sensor 501 may be disposed such that a surface of a sensing portion 502 exposed to a target component in a specimen gas is parallel to a direction in which the first flow path 103 passes the specimen gas (FIGS. 5A-5C), or gas sensor 501 may be disposed such that the surface of sensing portion 502 traverses (or is substantially perpendicular to) the direction in which first flow path 103 passes the specimen gas (FIG. 5D) to better expose the sensing portion to the target component. In the flow path the gas sensor can be set at a variety of levels, as shown in FIGS. 5A-5C. Preferably, the gas sensor's level is determined, with a condition of a gas containing a target component passing through the flow path (e.g., flow rate), the type of the target component, and the like taken into consideration, to satisfactorily expose the sensing portion to the target component. As a specific example, if a carrier gas flowing above the gas sensor flows at a slow rate, the gas sensor is positioned in the flow path at a lower level.

To allow the gas sensor to be adjusted in level in the flow path depending upon different conditions of a gas containing a target component passing through the flow path, different types of target components, and the like, the gas sensor is preferably movable. For example, the gas sensor may be adjusted in level automatically as controlled by the control unit. In other words, the present gas component detection device can be configured such that the switching operation by the flow path switching unit as well as the level adjustment of the gas sensor are automatically controlled by the control unit, once a certain target component has been selected.

Normally, the gas detection unit (or gas sensor) is connected via conductor or the like to signal receiving means such as a digital multimeter for receiving the signal variation that the gas detection unit shows (or variation in voltage of the constant resistance in the sensor). The gas component detection device may further include a computer connected to the signal receiving means. The computer stores detected signal data, converts the data to chromatogram, displays it, and the like. The computer may also serve as the control unit.

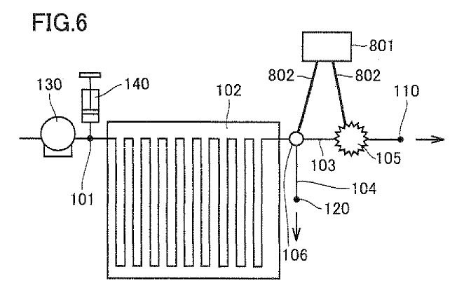

FIG. 6 schematically shows the present gas component detection device in another preferable example. The gas component detection device as shown in FIG. 6 includes a control unit 801 connected to gas detection unit 105 and flow path switching unit 106 by a conductor 802. Control unit 801 is for example a personal computer and gas detection unit 105 is connected to the personal computer via a digital multirneter (e.g., digital multimeter HP34401A available from Agilent Technologies (not shown in FIG. 6)). Control unit 801 stores detected signal data, converts the data to chromatogram, displays it, and the like. Furthermore, control unit 801 is also connected to flow path switching unit 106 and thus automatically controls a flow path switching operation. If the flow path switching operation is manually performed, the control unit may be connected only to the gas detection unit.

<Pump>

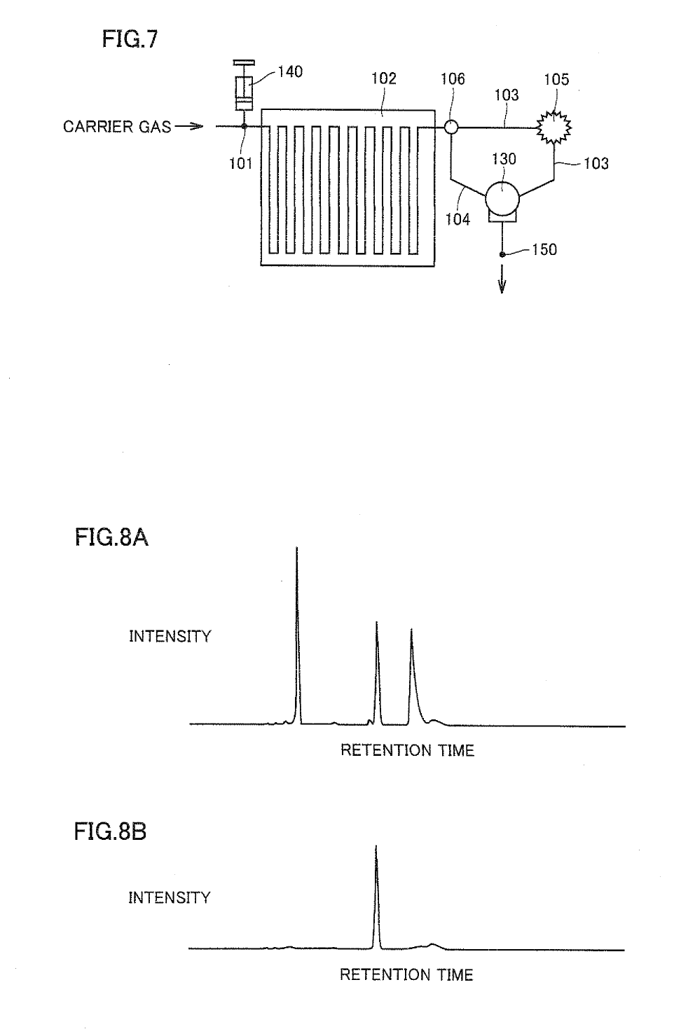

The pump operates to communicate a carrier gas (mobile phase) such as an inert gas (helium or the like) or air into the device and can be a conventionally known pump. The pump may be installed upstream of the gas separation unit, as shown in FIG. 1, or downstream of the gas separation unit, as shown in FIG. 7. For the latter example, the carrier gas is sucked by pump 130 and thus communicated through the device. If pump 130 is installed downstream of the gas separation unit, first flow path 103 and second flow path 104 are connected to pump 130, as shown in FIG. 7. A specimen gas which has passed through first flow path 103 or second flow path 104 passes through pump 130 and is discharged from gas discharging unit 150.

The present gas component detection device is susceptible of variations without departing from the gist of the present invention. For example, the gas component detection device as shown in FIG. 1 may have gas detection unit 105 in second flow path 104, rather than in first flow path 103. Alternatively, the gas component detection device may have both the gas detection unit in first flow path 103 and the gas detection unit in second flow path 104. This allows a plurality of target components in a specimen gas to be measured concurrently. Furthermore, the gas component detection device may have a distinct flow path branched in gas separation unit 102, a distinct flow path switching unit connected to the distinct flow path, distinct first and second flow paths, and a distinct gas detection unit. Such a configuration also allows a plurality of target components in a specimen gas to be measured concurrently.

### EXAMPLES

Hereinafter, examples will be presented to describe the present invention more specifically, though the present invention is not limited thereto.

### Example 1

The following procedure was followed to fabricate a gas component detection device having a configuration similar to FIG. 1. Initially, a microcoiumn of 4 cm square having a meandering internal flow path was fabricated as gas separation unit 102. A meandering groove of 100 μm in width and 150 μm in depth at intervals of 100 pin was formed by dry-etching on a surface of a Si wafer of 4 cm square, and subsequently on the grooved surface a glass plate of 4 cm square was bonded by anodic bonding and thus stacked thereon. An unmodified glass capillary having a diameter of 0.35 mm was attached to the bonded wafer and plate at the inlet and outlet ports of the internal flow path. Subsequently, a 0.6% pentane solution of 5% methyl phenyl silicone (Silicone SE-52 available from GL Sciences Inc.) was introduced into the internal flow path. The outlet port was sealed with a plug tightly, and to the inlet port a diaphragm type dry vacuum pump DA-15D (available from ULVAC KIKO Inc.) with a solvent trap attached thereto was connected. The pump was operated for several hours to completely remove the solvent in the internal flow path to fabricate the microcolumn. A rubber heater was attached at a lower portion of the microcolumn to allow temperature regulation.

A sampling pump SP208-100 Dual (available from GL Sciences Inc.) was used as pump 130. An adaptation of an introduction port employed for gas chromatography was used as gas introduction unit 101. A three way valve available from Swagelok Company was used as flow path switching unit 106. An acrylic chamber having a semiconductor sensor SB-30 (available from FIS Inc.) attached thereto was used as gas detection unit 105.

Gas separation unit 102 and gas introduction unit 101 were connected by using a ⅛×0.25 reducing union, and so were gas separation unit 102 and flow path switching unit 106. As seen in a direction in which a specimen gas flew, gas introduction unit 101, gas separation unit 102, and flow path switching unit 106 were connected such that unit 101 was upstream of unit 102 and unit 102 was upstream of unit 106, as shown in FIG. 7.

Furthermore, as shown in FIG. 7, flow path switching unit 106 and gas detection unit 105 were connected by a ⅛″ stainless steel pipe (first flow path 103), and flow path switching unit 106 and pump 130 were connected by a ⅛″ stainless steel pipe (second flow path 104). Furthermore, gas detection unit 105 and pump 130 were connected by a ⅛″ stainless steel pipe (first flow path 103). A gas component detection device was thus constructed.

The gas component detection device was employed to detect a gas component under conditions specifically as follows:

(1) specimen gas: mixture of ethanol, acetone and pentane;

(2) specimen gas introduced in an amount of: 1 μl;

(3) carrier gas: He at a flow rate of 10 mL/min; and

(4) microcolumn temperature: 40° C.

With flow path switching unit 106 connecting gas separation unit 102 to first flow path 103, an experiment was conducted under the above conditions to detect gas components. A chromatogram was obtained, as shown in FIG. 8A. In FIG. 5A, the peak for the shortest retention time corresponds to ethanol (retention time: approximately 2 minutes), the peak at the center corresponds to acetone (retention time: approximately 3 minutes), and the peak far the longest retention time corresponds to pentane (retention time: approximately 3.5 minutes).

Furthermore, with flow path switching unit 106 connecting gas separation unit 102 to second flow path 104, i.e., in an initial state, an experiment was conducted under the above conditions to detect gas components. In this experiment, a specimen gas was introduced and thereafter when a period of 2.5 minutes elapsed flow path switching unit 106 was switched to connect gas separation unit 102 to first flow path 103, and after the specimen gas was introduced when a period of 3.25 minutes elapsed flow path switching unit 106 was switched to again connect gas separation unit 102 to second flow path 104. A chromatogram was obtained, as shown in FIG. 8B. As shown in FIG. 8B, it has been confirmed that the flow path switching unit was appropriately switched to allow acetone to be alone guided to gas detection unit 105 and detected thereby.

### Example 2

A gas detection device was constructed in a manner similar to Example 1 except that the microcolumn having the meandering internal flow path as used in Example 1 was replaced with a microcolumn that was fabricated in the following method. A circular spiral groove of 100 μm in width and 150 μm in depth at intervals of 100 μm was formed by dry-etching on a surface of a Si wafer of 4 cm square and subsequently on the grooved surface a glass plate of 4 cm square was bonded by anodic bonding and thus stacked thereon to fabricate the microcolumn.

### Example 3

A gas detection device was constructed in a manner similar to Example 1 except that the microcolumn having the meandering internal flow path as used in Example 1 was replaced with a microcolumn that was fabricated in the following method. A squarely angled spiral groove of 100 μm in width and 150 μm in depth at intervals of 100 μm was formed by dry-etching on a surface of a Si wafer of 4 cm square and subsequently on the grooved surface a glass plate of 4 cm square was bonded by anodic bonding and thus stacked thereon to fabricate the microcolumn.

Although the present invention has been described and illustrated in detail, it is clearly understood that the same is by way of illustration and example only and is not to be taken by way of limitation, the scope of the present invention being interpreted by the terms of the appended claims.

What is claimed is:

1. A gas component detection device comprising:
a gas introduction unit for introducing a specimen gas;
a gas separation unit connected to said gas introduction unit;
a flow path switching unit connected to said gas separation unit and having a plurality of connection flow paths for switching a flow path that is connected to said gas separation unit to any one of said plurality of connection flow paths; and
a gas detection unit provided in at least one of said plurality of connection flow paths.

2. The gas component detection device according to claim 1, further comprising a control unit for controlling an operation performed by said flow path switching unit for switching.

3. The gas component detection device according to claim 2, wherein said control unit includes a storage unit having any preset time stored therein to control in accordance with said preset time said operation performed by said flow path switching unit for switching.

4. The gas component detection device according to claim 2, wherein said control unit includes a storage unit having stored therein any one or two or more preset gas components and a period(s) of time elapsing after said any one or two or more gas components are introduced through said gas introduction unit before said any one or two or more gas components are detected by said gas detection unit, and said operation performed by said flow path switching unit for switching is controlled in accordance with said period(s) of time of said any one or two or more gas components selected.

5. The gas component detection device according to claim 1, wherein said gas separation unit is formed of a chromatography column having therein a flow path having a width and depth of micro order.

6. The gas component detection device according to claim 5, wherein said chromatography column has said flow path in one of a meandering form, a squarely angled spiral form and a circular spiral form.

7. The gas component detection device according to claim 1, wherein said gas detection unit is a movable gas sensor.

8. The gas component detection device according to claim 1, wherein said specimen gas is exhaled breath and said gas introduction unit has a port receiving and introducing the exhaled breath.

9. The gas component detection device according to claim 1, wherein said specimen gas includes acetone.
